(19) United States
(12) Patent Application Publication
(10) Pub. No.: US 2017/0000389 A1
(43) Pub. Date: Jan. 5, 2017

(54) BIOMECHANICAL INFORMATION DETERMINATION

(71) Applicant: Colorado Seminary, which owns and operates the University of Denver, Denver, CO (US)

(72) Inventors: Bradley Davidson, Littleton, CO (US); Michael Decker, Parker, CO (US); Craig Simons, Boulder, CO (US); Kevin Shelburne, Golden, CO (US); Daniel Jung Kim, Aurora, CO (US)

(21) Appl. No.: 15/199,087

(22) Filed: Jun. 30, 2016

Related U.S. Application Data
(60) Provisional application No. 62/186,889, filed on Jun. 30, 2015.

(51) Int. Cl. A61B 5/11; A61B 5/00

(52) U.S. Cl. CPC A61B 5/1127; A61B 5/1114; A61B 5/6843; A61B 5/743; A61B 5/744; A61B 5/1112; A61B 5/0022; A61B 2562/0219; A61B 2562/028

(57) ABSTRACT

Systems and methods of the present disclosure may include initializing a first plurality of inertial measurement units (IMUs) and a second plurality of IMUs, and attaching the first plurality of IMUs to a first segment of a subject and the second plurality of IMUs to a second segment of the subject. Such a method may also include obtaining data from the first plurality of IMUs and the second plurality of IMUs as the subject performs a motion, and determining an absolute position of the first segment and the second segment based on the data.

### CROSS-REFERENCE TO RELATED APPLICATIONS

This application claims priority to U.S. Provisional Application No. 62/186,889, filed Jun. 30, 2015, titled BIOMECHANICS INFORMATION DETERMINATION, which is incorporated herein by reference in their entireties.

### SUMMARY

One or more embodiments of the present disclosure may include a method that may include recording first initial orientation information of a first inertial measurement unit (IMU) placed in a first initialization position at a first initialization location, and recording second initial orientation information of a second IMU placed in a second initialization position at a second initialization location. The method may also include placing the first IMU on a first segment of a subject, and placing the second IMU on a second segment of the subject, wherein the first segment and the second segment move relative to each other about a joint of the subject. The method may additionally include recording first acceleration information output by the first IMU in a continuous manner after recordation of the first initial orientation information of the first IMU, and recording second acceleration information output by the second IMU in the continuous manner after recordation of the second initial orientation information. The method may additionally include determining a first absolute location of the first segment with respect to the first initialization location based on the first acceleration information and the first initial orientation information. The method may also include determining a second absolute location of the second segment with respect to the second initialization location based on the second acceleration information and the second initial orientation information, and determining kinematics of the first segment and the second segment with respect to the joint based on the first absolute location and the second absolute location.

In accordance with one or more embodiments of the present disclosure, one or more methods of the present disclosure may additionally include recording first final orientation information of the first IMU at the first initialization location, determining a difference between the first final orientation and the first initial orientation information, and adjusting the first absolute location based on the difference.

In accordance with one or more embodiments of the present disclosure, one or more methods of the present disclosure may additionally include placing a third IMU on the first segment, recording third acceleration information output by the third IMU. Additionally, determining the first absolute location may further be based on the third acceleration information.

In accordance with one or more embodiments of the present disclosure, one or more methods of the present disclosure may additionally include comparing a first determination of the first absolute location based at least on the first acceleration information with a second determination of the first absolute location based at least on the third acceleration information, and correcting the first absolute location by an offset amount related to the comparison.

In accordance with one or more embodiments of the present disclosure, one or more methods of the present disclosure may additionally include placing a force sensor at a contact point on the subject, the force sensor configured to obtain force information with respect to pressure applied to a surface by the contact point. Additionally, biomechanical information of the first segment and the second segment with respect to the joint may be based on the force information.

One or more embodiments of the present disclosure may include a system that includes a first inertial measurement unit (IMU) attached to a first segment of a subject, and a second IMU attached to a second segment of the subject, where the first segment and the second segment move relative to each other about a joint of the subject. The system may additionally include a first force sensor attached to a first contact point of the subject, where the first force sensor may be attached to the first contact point such that the first force sensor is configured to obtain first pressure information with respect to pressure applied to a surface by the first contact point. The system may also include a second force sensor attached to a second contact point of the subject, where the second force sensor may be attached to the second contact point such that the second force sensor is configured to obtain second pressure information with respect to pressure applied to the surface by the second contact point. The system may additionally include a computing system communicatively coupled to the first IMU, the second IMU, the first force sensor, and the second force sensor. The computing system may be configured to obtain first acceleration information measured by the first IMU, obtain second acceleration information measured by the second IMU, obtain first pressure information measured by the first force sensor, and obtain second pressure information measured by the second force sensor. The computing system may also be configured to determine kinetics of the subject with respect to the joint based on the first acceleration information, the second acceleration information, the first pressure information, and the second pressure information.

In accordance with one or more embodiments of the present disclosure, a computing system may additionally be configured to determine the kinetics with respect to one or more of the following: a time when both the first contact point and the second contact point are applying pressure to the surface, a time when the first contact point is applying pressure to the surface and the second contact point is not applying pressure to the surface, and a time when the second contact point is applying pressure to the surface and the first contact point is not applying pressure to the surface.

In accordance with one or more embodiments of the present disclosure, a system may additionally include a first plurality of IMUs attached to the first segment and a second plurality of IMUs attached to the second segment.

One or more embodiments of the present disclosure may include a method that may include initializing a first plurality of inertial measurement units (IMUs) and a second plurality of IMUs, and attaching the first plurality of IMUs to a first segment of a subject and the second plurality of IMUs to a second segment of the subject. Such a method may also include obtaining data from the first plurality of IMUs and the second plurality of IMUs as the subject performs a motion, and determining an absolute position of the first segment and the second segment based on the data.

In accordance with one or more embodiments of the present disclosure, the first plurality of IMUs are attached to the subject before initializing the first plurality of IMUs.

In accordance with one or more embodiments of the present disclosure, initializing the first plurality of IMUs may include obtaining a plurality of images, each of the first plurality of IMUs being in one or more of the plurality of images, and displaying at least one of the plurality of images. Initializing the first plurality of IMUs may additionally include identifying one or more joints of the subject in the at least one of the plurality of images, projecting a skeletal model over the subject in the at least one of the plurality of images, and overlaying a geometric shape over the at least one of the plurality of images, the geometric shape corresponding to the first segment.

In accordance with one or more embodiments of the present disclosure, one or more methods of the present disclosure may additionally include providing a prompt to identify one or more joints of the subject in the at least one of the plurality of images, and receiving an identification of one or more joints of the subject.

In accordance with one or more embodiments of the present disclosure, one or more methods of the present disclosure may additionally include providing a prompt to input anthropometric information, and receiving anthropometric information of the subject. Additionally, at least one of the skeletal model and the geometric shape may be based on the anthropometric information of the subject.

In accordance with one or more embodiments of the present disclosure, one or more methods of the present disclosure may additionally include providing a prompt to adjust the geometric shape to align the geometric shape with an outline of the subject, receiving an input to adjust the geometric shape, and adjusting the geometric shape based on the input.

In accordance with one or more embodiments of the present disclosure, one or more methods of the present disclosure may additionally include obtaining global positioning system (GPS) location of an image capturing device, and capturing at least one of the plurality of images using the image capturing device.

In accordance with one or more embodiments of the present disclosure, one or more methods of the present disclosure may additionally include placing the image capturing device in a fixed location of a known position, and where the GPS location of the image capturing device is the fixed location.

In accordance with one or more embodiments of the present disclosure, capturing at least one of the plurality of images may additionally include capturing a plurality of images using a plurality of image capturing devices such that each IMU of the first plurality of IMUs is in at least two of the plurality of images.

In accordance with one or more embodiments of the present disclosure, capturing at least one of the plurality of images may additionally include capturing a video of the subject, the video capturing each of the first plurality of IMUs.

In accordance with one or more embodiments of the present disclosure, one or more methods of the present disclosure may additionally include determining an image-based absolute position of the first segment based on the GPS location of the image capturing device, and modifying the absolute position based on the image-based absolute position.

In accordance with one or more embodiments of the present disclosure, initializing the IMUs may additionally include performing a three-dimensional scan of the subject.

### BRIEF DESCRIPTION OF THE DRAWINGS

Example embodiments will be described and explained with additional specificity and detail through the use of the accompanying drawings in which:

FIG. 1 illustrates an example system for determining biomechanical information of a subject;

FIGS. 2A-2D illustrate various examples of placement of sensors on a subject;

FIG. 3 illustrates a block diagram of an example computing system;

FIG. 4 illustrates a flowchart of an example method for determining biomechanical information of a subject; and

FIG. 5 illustrates a flowchart of an example method for initializing one or more sensors.

### DETAILED DESCRIPTION

Some embodiments described in the present disclosure relate to methods and systems of determining biomechanical information of a subject, e.g., a person or an animal. One or more inertial measurement units (IMUs) may be initialized and attached to a subject. The subject may then perform a series of motions while data from the IMUs is collected. After the series of motions is performed, the data may be analyzed to provide kinematic information regarding the subject. In some embodiments, low cost IMUs may be used and multiple IMUs may be attached to each body segment being analyzed to facilitate accurate readings for reliable kinematic information.

The biomechanical information may include information related to kinematics and/or kinetics with respect to one or more joints of the subject. Kinematics may include motion of segments of the subject that may move relative to each other about a particular joint. The motion may include angles of the segments with respect to an absolute reference frame (e.g., the earth), angles of the segments with respect to each other, etc. The kinetics may include joint moments and/or muscle forces, and/or joint forces that may be a function of the kinematics of the corresponding segments and joints.

As detailed in the present disclosure, systems and methods are described in which IMUs may be attached to a subject to gather information that may be used to determine biomechanical information. Many have rejected using IMUs to determine biomechanical information because of inaccuracies in the information determined from IMUs. However, the use of IMUs in the manner described in the present disclosure may be more accurate than other techniques and may allow for biomechanical determinations and measurements to be made using IMUs. The use of IMUs may expand the ability to measure and determine biomechanics outside of a laboratory and in unconstrained settings.

While the present disclosure uses IMUs as an example type of sensor used to derive biomechanical information, any type of sensor may be used in accordance with principles of the present disclosure and be within the scope of the present disclosure. For example, a micro-electro-mechanical system (MEMS) or other sensor that may be attached to a subject and that may measure similar or analogous information as an IMU is also within the scope of the present disclosure. Thus, in some embodiments, any of a variety of sensors or combinations thereof may be attached to a user to facilitate determination of biomechanical information. Additionally or alternatively, in some embodiments, sensors may be included to monitor and/or measure one or more physiological characteristics of the user, such as heart rate, blood pressure, blood oxygenation, etc. Some examples of sensors that may be coupled to the user may include a gyroscope, an accelerometer, a speedometer, a potentiometer, a global positioning system sensor, a heart rate monitor, a blood oxygen monitor, electromyocardiogram (EMG), etc.

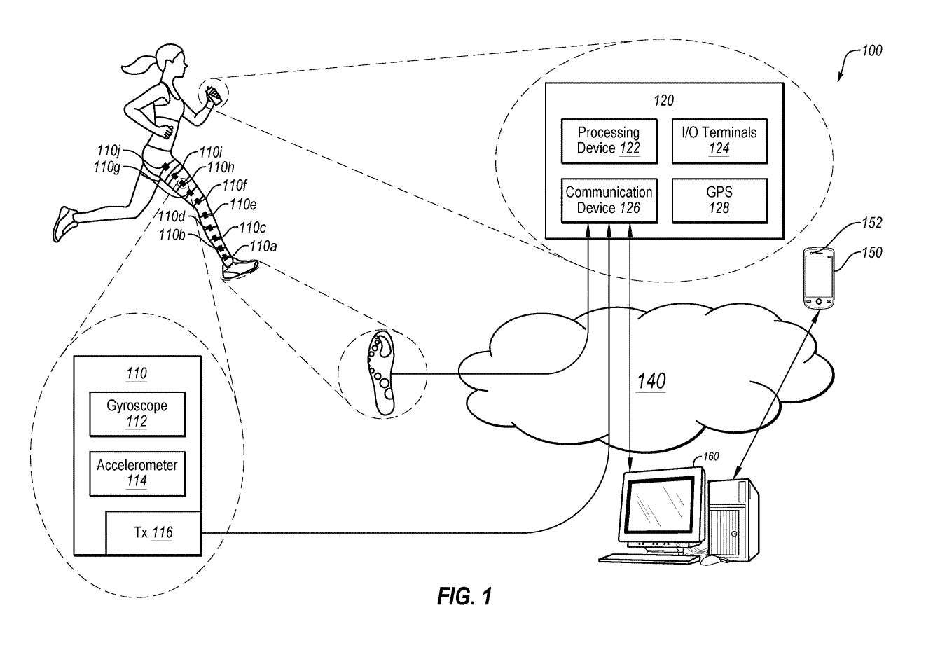

FIG. 1 illustrates an example system 100 for determining biomechanical information of a subject, in accordance with one or more embodiments of the present disclosure. The system 100 may include one or more sensors, such as an IMU 110, disposed at various locations about a subject. As illustrated in FIG. 1, the system 100 may include IMUs 110a-110j (which may be referred to collectively as the IMUs 110). The IMUs 110 may be configured to capture data regarding position, velocity, acceleration, magnetic fields, etc. and may be in communication with a computing device 120 to provide the captured data to the computing device 120. For example, the IMUs 110 may communicate with the computing device 120 over the network 140.

In some embodiments, the IMUs 110 may include sensors that may be configured to measure acceleration in three dimensions, angular speed in three dimensions, and/or trajectory of movement in three dimensions. The IMUs 110 may include one or more accelerometers 114, one or more gyroscopes 112, and/or one or more magnetometers to make the above-mentioned measurements. Examples and descriptions are given in the present disclosure with respect to the use of IMUs 110 attached to a subject to obtain information about the subject. However, any other micro-electro-mechanical system (MEMS) or other sensor that may be attached to a subject and that may measure similar or analogous information as an IMU is also within the scope of the present disclosure.

In some embodiments, a calibration technique may be employed to improve the accuracy of information that may be determined from the IMUs 110, and/or to initialize the IMUs 110. For example, in some embodiments, initial orientation information of the IMUs 110 that may be placed on segments of a subject may be determined. The initial orientation information may include information regarding an initial orientation of the IMUs 110 at an initial location. The initial orientation information may be determined when the IMUs 110 are each in a known initial orientation at a known initial location. In some embodiments, the known initial orientation, and the known initial locations may be used as initial reference points of an absolute reference frame that may be established with respect to the known initial locations.

In some embodiments, the known initial location may be the same for one or more of the IMUs 110 and in some embodiments may be different for one or more of the IMUs 110. Additionally or alternatively, in some embodiments, as discussed in further detail below, the known initial locations may be locations at which one or more IMUs 110 are attached to a segment of the subject. In these or other embodiments, the known initial orientations and known initial locations of the IMUs 110 may be based on the orientations of the corresponding segments to which the IMUs 110 may be attached when the subject has the corresponding segment in a particular position at a particular location.

Additionally or alternatively, a calibration fixture (e.g., a box or tray) may be configured to receive the IMUs 110 in a particular orientation to facilitate initializing the IMUs 110. In these or other embodiments, the location of the calibration fixture and a particular IMU (e.g., the IMU 110a) within the calibration fixture at the time that the initial orientation information is obtained may serve as the initial location for the particular IMU. In some embodiments, the calibration fixture may include multiple receiving portions each configured to receive a different IMU at a particular orientation (e.g., the IMUs 110a and 110b). In these or other embodiments, initial orientation information may be obtained for multiple IMUs (e.g., the IMUs 110a and 110b) that may each be placed in the same receiving portion at different times. In some embodiments, the calibration fixture may include any suitable system, apparatus, or device configured to establish its position and orientation in an absolute reference frame. For example, in some embodiments, the calibration fixture may include one or more Global Navigation Satellite System (GNSS) sensors (e.g., one or more GPS sensors) and systems configured to establish the position and orientation of the calibration fixture in a global reference frame (e.g., latitude and longitude).

In some embodiments, after the IMUs 110 have been initialized (e.g., after initial orientation information has been determined), acceleration information may be obtained from the IMUs 110 while the subject performs one or more motions. As used herein, when a user is described as performing one or more motions, such motions may include holding a given posture, or holding a segment in a given posture such that the user may or may not actually move certain segments of the user's body. In some embodiments, the acceleration information may be obtained in a continuous manner. The continuous manner may include obtaining the acceleration information in a periodic manner at set time intervals. In some embodiments, the acceleration information may be integrated with respect to time to determine velocity information and the velocity information may be integrated with respect to time to obtain distance information. The distance information and trajectory information with respect to the acceleration information may be used to determine absolute locations of the IMUs 110 with respect to their respective known initial locations at a given time.

In these or other embodiments, the absolute locations and known initial locations may be used to determine relative locations of the IMUs 110 with respect to each other. Additionally or alternatively, orientation information that corresponds to an absolute location at a given time may be used to determine relative locations of the IMUs 110 with respect to each other. In some embodiments, acceleration and gyroscope information may be fused via an algorithm such as, for example, a Complementary filter, a Kalman Filter, an Unscented Kalman Filter, an Extended Kalman Filter, a Particle Filter, etc., to determine the orientation information.

In some embodiments, the relative locations of the IMUs 110 (e.g., based on the orientation information) may be used to audit or improve the absolute location determinations.

For example, the relative locations of the IMUs 110 that may be determined based on the orientation information may be compared with the relative locations that may be determined based on the absolute locations. In these or other embodiments, the absolute location determinations may be adjusted based on the comparison.

In some embodiments, continuous acceleration measurements may be made while the IMUs 110 are attached to segments of the subject and/or while the IMUs 110 are removed from their initial locations within the calibration fixture for attachment to the respective segments. Therefore, absolute and relative location determinations of the IMUs 110 may be used to determine absolute and relative positions of the respective segments. In these or other embodiments, joint orientation may be determined based on the absolute and relative location determinations. In these and other embodiments, multiple absolute and relative location determinations of the segments and multiple joint orientation determinations may be used to determine biomechanical information of the respective segments with respect to the corresponding joints, as discussed in the present disclosure. The IMUs 110 may be attached to the corresponding segments using any suitable technique and any suitable location and orientation on the segments. In some embodiments, the location data may be associated with timestamps that may be compared with when the IMUs 110 are attached to a subject to differentiate between times when the IMUs 110 may be attached to the subject and not attached to the subject.

In these or other embodiments, one or more GNSS sensors may be attached to the subject and accordingly used to determine an approximation of the absolute location of the subject. The approximation from the GNSS sensors may also be used to adjust or correct the absolute location that may be determined from the IMUs 110 acceleration information. The adjustment in the absolute location determinations may also be used to adjust corresponding biomechanics determinations. In some embodiments, the GNSS sensors may be part of the computing device 120. For example, the GNSS sensors may be part of a GPS chip 128 of the computing device 120.

In these or other embodiments, the IMUs 110 may be returned to their respective initial locations after different location measurements have been determined while the subject has been moving with the IMUs 110 attached. Based on the acceleration information, an absolute location may be determined for the IMUs 110 when the IMUs 110 are again at their respective initial locations. Additionally or alternatively, if the absolute locations are not determined to be the same as the corresponding initial locations, the differences may be used to correct or adjust one or more of the absolute location determinations of the IMUs 110 that may be made after the initial orientation information is determined. The adjustment in the absolute location determinations may also be used to adjust corresponding kinematics determinations.

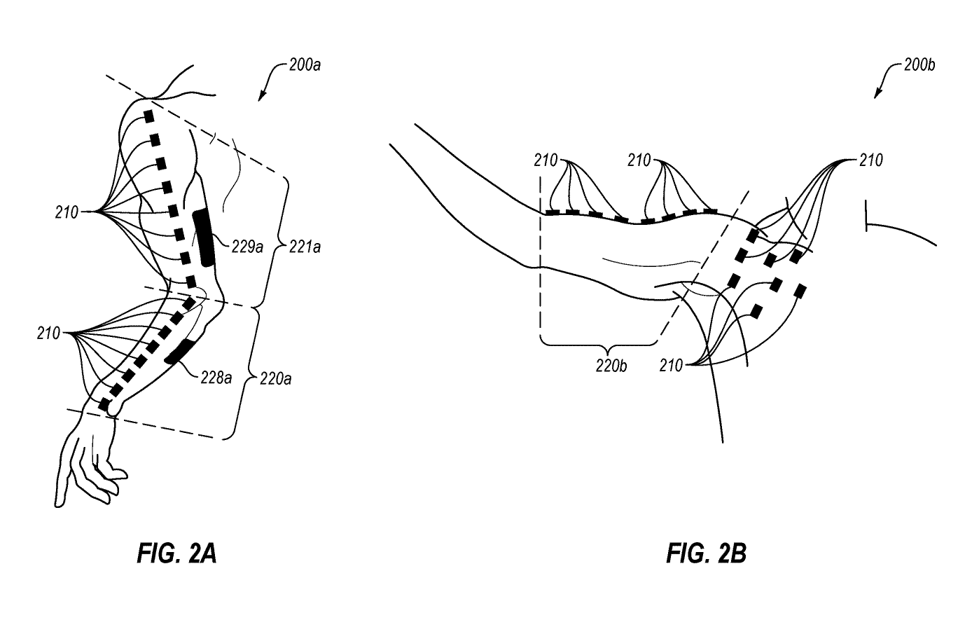

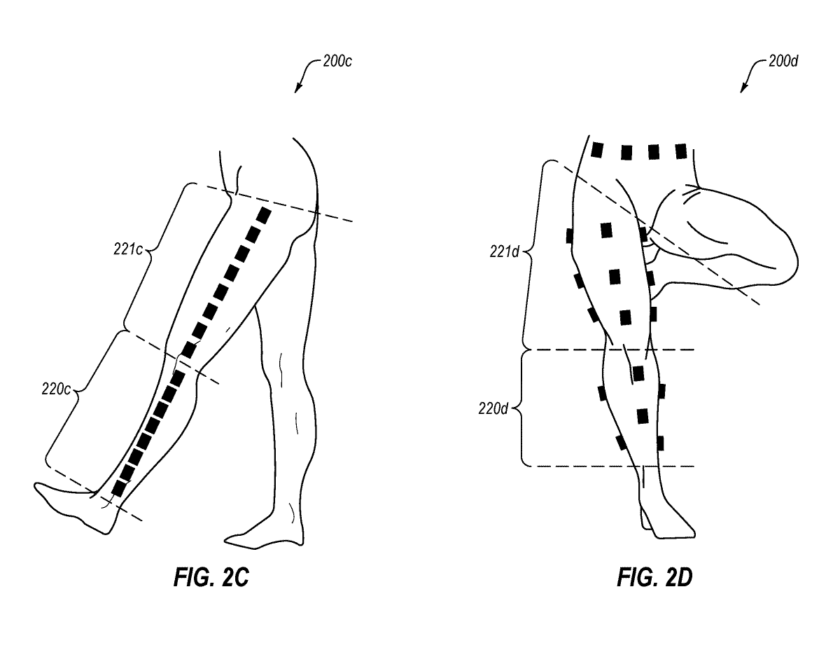

In some embodiments, the number of IMUs 110 per segment, the number and type of segments that may have IMUs 110 attached thereto, and such may be based on a particular portion of the body of the subject that may be analyzed. Further, the number of IMUs 110 per segment may vary based on target biomechanical information that may be obtained. Moreover, in some embodiments, the number of IMUs 110 per segment may be based on a target accuracy in which additional IMUs 110 per segment may provide additional accuracy. For example, the data from different IMUs 110 attached to a same segment may be compared and differences may be resolved between the different IMUs 110 to improve kinematics information associated with the corresponding segment. For example, a common angular velocity of the segment may be determined across multiple IMUs 110 for a single segment. In some embodiments, any number of IMUs 110 may be attached to a single segment, such as between one and five, one and ten, one and fifteen, etc. Examples of some orientations and/or number of IMUs 110 attached to a subject may be illustrated in FIGS. 2A-2D.

In some embodiments, calibration techniques and/or correction approaches may be based on iterative approaches and/or a combination of corrective approaches (e.g., a Complementary filter, a Kalman Filter, an Unscented Kalman Filter, an Extended Kalman Filter, a Particle Filter, etc.). For example, measuring a particular variable (e.g., absolute location) with two different calculation methods of which each method contains a unique estimation error may be fused together with iterative steps until convergence between the two possible solutions is reached. Such an approach may yield a more accurate estimation of the particular variable than either of the calculation methods on their own. In these and other embodiments, various motions (including, e.g., poses, duration of poses, etc.) may be performed and/or repeated to gather sufficient data to perform the various calculation approaches. Such a process of estimating and correcting for measurement error may yield a superior result to a Kalman filter on its own.

In some embodiments, the calibration techniques, location determinations (absolute and/or relative), and associated biomechanical information determinations may be made with respect to an anthropometric model of the subject. For example, the anthropometric model may include height, weight, segment lengths, joint centers, etc. of the subject. In some embodiments, anthropometric information of the subject may be manually entered, automatically detected, or selected from an array of options. Further, in some embodiments, locations of the IMUs 110 on the segments of the subject may be included in the model. In these or other embodiments, the kinematics of the segments may be determined based on the locations of the IMUs 110 on the segments. For example, if five IMUs 110 were disposed between the ankle and the knee of the subject, and five additional IMUs 110 were disposed between the knee and the hip of the subject, kinematics and/or other biomechanical information regarding the knee joint of the subject may be observed and/or derived. Such information may include joint angle, joint moments, joint torques, joint power, muscle forces, etc.

In some embodiments, the calibration described above may include optical reference localization that may be used to determine reference locations for determining the absolute locations of the IMUs 110 and accordingly of the segments of the subject. For example, in some embodiments, the reference locations may include locations of the IMUs 110 when the IMUs 110 are attached to a particular segment of the subject and when the particular segment is in a particular position. In these or other embodiments, the optical reference localization technique may include a triangulation of optical information (e.g., photographs, video) taken of the subject with the IMUs 110 attached to the segments in which the locations of the optical capturing equipment (e.g., one or more cameras) may be known with respect to the initial locations.

In some embodiments, the optical information may be obtained via an image capturing device 150. The image capturing device 150 may include a camera 152. The image capturing device 150 may include position sensing components, such as a GPS chip or other components to determine the location of the image capturing device 150 when the image is captured or to determine the distance from the image capturing device 150 to the subject. In these and other embodiments, with triangulation and/or known locations of the image capturing device 150 with respect to the initial locations, the reference locations of the IMUs 110 may be determined. The optical reference localization may be performed using any suitable technique, various examples of which are described in the present disclosure.

In some embodiments, the locations of the image capturing device 150 with respect to the initial locations may be determined based on simple distance and direction measurements or GNSS (e.g., GPS coordinates). In these or other embodiments, image capturing device 150 may include one or more IMUs 110 or may have one or more IMUs 110 attached thereon. For example, the image capturing device 150 may include a wireless electronic device such as a tablet computer or a smartphone. In these and other embodiments, the location of the image capturing device 150 that may be used to obtain optical information of the subject may be determined by first placing the image capturing device 150 at a particular orientation in the calibration fixture and determining the location of the image capturing device 150 based on acceleration information of a corresponding IMU 110.

In some embodiments, the reference locations may be used as initial locations. In these or other embodiments, the known locations of the image capturing device 150 may be based on a particular coordinate system and the initial locations may include the reference locations as determined with respect to the particular coordinate system. For example, the locations of the image capturing device 150 may be known with respect to a global reference system (e.g., latitude and longitude) based on GNSS information. In these or other embodiments, the determined reference locations may be determined based on the GNSS information and optical reference localization and may be used as initial locations.

As another example, the locations of the image capturing device 150 may be known within a room and a coordinate system that may be established with respect to the room. In these or other embodiments, the determined reference locations may be identified based on the room coordinate system, the known locations of the image capturing device 150 with respect to the room coordinate system and the triangulation. In some embodiments, the optical reference localization may also be used to apply a particular anthropometric model to a particular subject.

Listed below are some examples of performing optical reference localization with respect to calibration and/or initialization of the IMUs 110 for a particular subject. The techniques listed below are not meant to be limiting.

According to a first technique, one or more fixed image capture devices 150 may be used. Using such a technique, a user may select a particular musculoskeletal model (e.g. lower extremity only, lower extremity with torso, full body, Trendelenburg, etc.). In these and other embodiments, each model may have a minimum number of IMUs 110 associated with the model chosen. Multiple image capture devices 150 may be located at known distances from a capture volume where the subject is located (e.g., 2-3 web cameras may be disposed one meter away from the subject). One or more synchronous snapshots of the subject may be taken from the multiple image capture devices 150. One or more of the captured images may then be displayed simultaneously on a computing device, such as the computing device 160. A user of the computing device 160 may be prompted to indicate locations of joint centers of the model chosen (ankle, knees, hips, low back, shoulders, etc.). For example, the user may be provided with a selection tool via a user interface at the computing device 160 via which the user may indicate the location of one or more of the join centers in the image(s) displayed at the computing device 160. Additionally or alternatively, the user of the computing device 160 may be prompted to indicate locations in each image of each IMU associated with the chosen skeletal model. After identifying the joint centers and/or the IMUs, a skeletal model may be projected onto one or more of the images. The user may be prompted to input anthropometric information regarding the subject, and one or more of the skeletal models may be adjusted accordingly. In these and other embodiments, a geometric volume (e.g., an ellipsoid or frustum) for one or more segments may be overlaid onto an image. Geometric dimensions of the geometric volume may be based on joint center selection and/or anthropometric information (e.g., the height and/or weight of the subject). In these and other embodiments, the user of the computing device 160 may be prompted to adjust the geometric volume size to match the segment in the image.

According to a second technique, one or more movable image capture devices 150 may be used. Using the second technique, a user may place and/or retrieve the image capturing device 150 from a known location (e.g., a calibration fixture similar and/or analogous to that used in initializing IMUs). The image capturing device 150 may be used to capture multiple images of the subject such that each of the IMUs 110 and/or each of the joint centers associated with the IMUs 110 may be in two or more images. In some embodiments, the subject may remain in a fixed position or stance while the images are captured. One or more of the captured images may be associated with a time stamp of when the image was captured, and one or more of the captured images may then be displayed simultaneously on a computing device, such as the computing device 160. A user of the computing device 160 may be prompted to indicate locations of joint centers of a chosen model (ankle, knees, hips, low back, shoulders, etc.). For example, the user may be provided with a selection tool via a user interface at the computing device 160 via which the user may indicate the location of one or more of the join centers in the image(s) displayed at the computing device 160. Additionally or alternatively, the user of the computing device 160 may be prompted to indicate locations of the IMUs associated with the chosen skeletal model. After identifying the joint centers and/or the IMUs, a skeletal model may be projected onto one or more of the images. The user may be prompted to input anthropometric information regarding the subject, and one or more of the skeletal models may be adjusted accordingly. In these and other embodiments, a geometric volume (e.g., an ellipsoid or frustum) for one or more segments may be overlaid onto an image. Geometric dimensions of the geometric volume may be based on joint center selection and/or anthropometric information (e.g., the height and/or weight of the subject). In these and other embodiments, the user of the computing device 160 may be prompted to adjust the geometric volume size to match the segment in the image.

According to a third technique, one or more movable image capture devices 150 capable of capturing video (e.g., a smart phone) may be used. This third technique may be similar or comparable to the second technique. However, rather than capturing images, the image capturing device 150 may capture video of the subject as the image capturing 150 is moved around the subject. Each of the still images of the video may be associated with a time stamp. Using the individual still images of the video with the time stamps, the third technique may proceed in a similar manner to the second technique.

According to a fourth technique, one or more movable image capture devices 150 capable of capturing video (e.g., a smart phone) may be used in addition to a three-dimensional (3D) scanner, such as an infrared scanner or other scanner using radiation at other frequencies. Using the fourth approach, a user may place and/or retrieve the image capturing device 150 from a known location (e.g., a calibration fixture). Using the image capturing device 150 and a 3D scanner, the user record video and a 3D scan of the subject that captures all the locations of the IMUs 110. In some embodiments, the 3D scanner may include a handheld scanner. In these or other embodiments, the 3D scanner may be combined with or attached to another device such as a tablet computer or smartphone. The 3D image from the scanner may be separated into multiple viewing planes. In some embodiments, at least three of the viewing planes may be oblique viewing planes (e.g., not cardinal planes). One or more depth images from one or more of the planes may be displayed simultaneously on the computing device 160. The user of the computing device 160 may be prompted to indicate locations in the planar views of each IMU associated with a chosen skeletal model. After identifying the joint centers and/or the IMUs, a skeletal model may be projected onto one or more of the images. The user may be prompted to input anthropometric information regarding the subject, and one or more of the skeletal models may be adjusted accordingly. In these and other embodiments, a geometric volume (e.g., an ellipsoid or frustum) for one or more segments may be overlaid onto an image. Geometric dimensions of the geometric volume may be based on joint center selection and/or anthropometric information (e.g., the height and/or weight of the subject). In these and other embodiments, the user of the computing device 160 may be prompted to adjust the geometric volume size to match the segment in the image.

Additionally or alternatively, in some embodiments, optical reference localization may be performed periodically to determine the absolute locations of the IMUs 110 that may be attached to the subject at different times. For example, if the subject were going through a series of exercises, the IMUs 110 may be reinitialized and/or the reference location verified periodically throughout the set of exercises. The absolute locations that may be determined from the optical reference localization may also be compared with the absolute locations determined from the IMU 110 acceleration information. The comparison may be used to adjust the absolute locations that may be determined from the IMU 110 acceleration information. The adjustment in the absolute location determinations may also be used to adjust corresponding biomechanical information.

In some embodiments, the correction and/or calibration may include any combination of the approaches described in the present disclosure. For example, multiple IMUs 110 may be attached to a single segment of a subject, and each of those IMUs 110 may be initialized using the image capturing device 150 by taking a video of the subject that captures each of the IMUs 110. After a first set of exercises, the IMUs 110 may be reinitialized using the image capturing device 150 to capture an intermediate video. After the exercises are completed, the IMUs 110 may again be captured in a final video captured by the image capturing device 110. The absolute location of the segment may be based on data from the IMUs 110 corrected based on the multiple IMUs 110 attached to the segment and corrected based on the intermediate video and the final video.

In some embodiments, the localization determinations and anthropometric model may be used to determine biomechanical information of the segments with respect to corresponding joints. For example, the localization (e.g., determined absolute and relative locations), linear velocity, and linear acceleration of segments may be determined from the acceleration information as indicated in the present disclosure to determine inertial kinematics with respect to the segments. Further, the anthropometric model of the subject may include one or more link segment models that may provide information on segment lengths, segment locations on the subject, IMU locations on the segments, etc. The determined inertial kinematics may be applied to the link segment model to obtain inertial model kinematics for the segments themselves.

In some embodiments, the kinematics determinations may be used to determine other biomechanical information, such as kinetics, of the subject. For example, in some embodiments, the kinematics determinations may be used to determine kinetic information (e.g., joint moments, joint torques, joint power, muscle forces, etc.) with respect to when a single contact point (e.g., one foot) of the subject applies pressure against a surface (e.g., the ground). In these or other embodiments, information from a force sensor 130 (e.g., insole pressure sensors) attached to the subject may be obtained. The force information in conjunction with the determined kinematics for the segments, and the determined joint orientation may be used to determine kinetic information. In some embodiments, inverse dynamics may be applied to the localization information and/or the force information to determine the biomechanical information.

Additionally or alternatively, in some embodiments, the pressure information may be used in determining kinetic information when more than one contact point of the subject is applying pressure to a surface based on comparisons between pressure information associated with the respective contact points applying pressure against the surface. For example, comparisons of pressure information from the force sensors 130 associated with each foot may be used to determine kinetic information with respect to a particular leg of the subject at times when both feet are on the ground.

In some embodiments, machine learning techniques may be used to improve the accuracy of the localization determinations and/or the force determinations. Additionally or alternatively, the machine learning techniques may be used to infer additional information from the localization and/or force determinations. For example, the machine learning may be used to infer force parallel to a surface from force information that is primarily focused on force perpendicular to the surface. In these or other embodiments, the machine learning techniques may be used to augment or improve kinetics determinations by making inferences with respect to the kinetic information.

By way of example, the machine learning techniques may include one or more of the following: principal component analysis, artificial neural networks, support vector regression, etc. In these or other embodiments, the machine learning techniques may be based on a particular activity that the subject may be performing with respect to the localization and/or pressure information.

In these and other embodiments, the IMUs 110 and/or the force sensor 130 may provide any captured data or information to the computing device 120. For example, the IMUs 110 and/or the force sensor 130 may continuously capture data readings and may transmit those data readings to be stored on the computing device 120. In these and other embodiments, the computing device 120 may utilize the obtained data, or may provide the data to another computing device to utilize (e.g., the computing device 160). The IMUs 110 may include a transmitting device 116 for providing the data to the computing device 120. The force sensor 130 may include a similar transmitting component. The computing device 120 may include a processing device 122 for controlling operation of the computing device 120, a communication device 126 for communicating with one or more of the IMUs 110, the force sensor 130, and the computing device 160, input/output (I/O) terminals 124 for interacting with the computing device 120, and/or the GPS chip 128.

The network 140 may facilitate communication between any of the IMUs 110, the computing device 120, the force sensor 130, the image capturing device 150, and/or the computing device 160. The network 140 may include Bluetooth connections, near-field communications (NFC), an 802.6 network (e.g. Metropolitan Area Network (MAN)), WiFi network, WiMax network, cellular network, a Personal Area Network (PAN), an optical network, etc.

In some embodiments, the computing device 120 may be implemented as a small mobile computing device that can be held, worn, or otherwise disposed about the subject such that the subject may participate in a series of motions without being inhibited. For example, many individuals carry a smartphone or tablet about their person throughout most of the day, including when performing exercise. In these and other embodiments, the computing device 120 may be implemented as a smartphone, a tablet, a Raspberry Pi®, etc. In some embodiments, the computing device 120 may provide collected data to the computing device 160. In these and other embodiments, the computing device 160 may have superior computing resources, such as processing speed, storage capacity, available memory, or ease of user interaction.

In some embodiments, multiple components illustrated as distinct components in FIG. 1 may be implemented as a single device. For example, the computing device 120 and the computing device 160 may be implemented as the same computing device. As another example, the image capturing device 150 may be part of the computing device 120 and/or the computing device 160.

Modifications, additions, or omissions may be made to the system 100 without departing from the scope of the present disclosure. For example, in some embodiments, the system 100 may include any number of other components that may not be explicitly illustrated or described. As another example, any number of the IMUs 110 may be disposed along any number of segments of the subject and in any orientation. As an additional example, the computing device 120 and/or the IMUs 110 may include more or fewer components than those illustrated in FIG. 1. As an additional example, any number of other sensors (e.g., to measure physiological data) may be included in the system 100.

FIGS. 2A-2D illustrate various examples of placement of sensors on a subject, in accordance with one or more embodiments of the present disclosure. FIG. 2A illustrates the placement of various sensors about an arm of a subject for analyzing an elbow joint, FIG. 2B illustrates the placement of various sensors about an upper arm and chest of a subject for analyzing an elbow joint, FIG. 2C illustrates the placement of various sensors about a leg of a subject for analyzing a knee joint, and FIG. 2D illustrates the placement of various sensors about a leg and abdomen of a subject for analyzing a knee joint and a hip joint. The FIGS. 2A-2D may also serve to illustrate examples of a user interface that may be provided to a user of a computing system at which the user may input the location of joint centers and/or the location of various sensors on a subject. For example, a user of the computing device 160 of FIG. 1 may be provided with a display comparable to that illustrated in FIG. 2A and asked to identify the center of a joint of interest and the location of various sensors.

As illustrated in FIG. 2A, in some embodiments, multiple IMUs 210 may be disposed along the arm of a subject. For example, a first segment 220a may include eight IMUs 210 placed in a line running the length of the first segment 220a. Additionally, a second segment 221a may include eight IMUs 210 in a line running the length of the second segment 221a. In these and other embodiments, the IMUs 210 may be placed directly along a major axis of the segment.

In some embodiments, a first GPS sensor 228a may be placed on the first segment 220a and a second GPS sensor 229a may be placed on the second segment 221a. In these and other embodiments, the first GPS sensor 228a may be utilized to facilitate determination of the absolute location of the first segment 220a and/or calibration or correction of the absolute location of the first segment 220a based on data from the IMUs 210. While described with respect to the first GPS sensor 228a and the first segment 220a, the same description is applicable to the second segment 221a and the second GPS sensor 229a.

In some embodiments, one or more of the sensors (e.g., the IMUs 210 and/or the first or second GPS sensors 228a, 229a) may be attached to the subject in any suitable manner. For example, the sensors may be disposed upon a sleeve or other tight-fitting clothing material that may then be worn by the subject. As another example, the sensors may be strapped to the subject using tieable or cinchable straps. As an additional example, the sensors may be attached to the subject using an adhesive to attach the sensors directly to the skin of the subject. The sensors may be attached individually, or may be attached as an array to maintain spacing and/or orientation between the various sensors.

As illustrated in FIG. 2B, eight IMUs 210 may be disposed along an upper arm of a subject in a first segment 220b, and eight IMUs 210 may be disposed around a chest of the subject. In some embodiments, the IMUs 210 on the chest of the subject may be disposed in a random or otherwise dispersed manner about the chest such that minor movements or other variations in the location of the chest relative to the shoulder joint may be accounted for in the biomechanical information derived regarding the shoulder joint.

As illustrated in FIG. 2C, eight IMUs 210 may be disposed along a first segment 220c along the lower leg of a subject, and eight IMUs 210 may be disposed along a second segment 221c along the upper leg of the subject. In some embodiments, the IMUs 210 may be disposed in a line along a major axis of the respective segments, similar to that illustrated in FIG. 2A. In these and other embodiments, the IMUs 210 may follow along a location of a bone associated with the segment. For example, the IMUs 210 of the first segment 220c may follow the tibia and the IMUs 210 of the second segment 221c may follow the femur.

As illustrated in FIG. 2D, six IMUs 210 may be disposed in a first segment 220d about the lower leg of a subject, nine IMUs 210 may be disposed about the upper leg of the subject, and four IMUs 210 may be disposed about the abdomen of the subject. As illustrated in FIG. 2D, in some embodiments, the IMUs 210 may be disposed radially around the outside of a particular segment of the subject. With reference to the first segment 220d, the IMUs 210 may be offset from each other when going around the circumference of the first segment 220d. With reference to the second segment 221d, the IMUs 210 may be aligned about the circumference of the second segment 221d.

As illustrated in FIGS. 2A-2D, various sensors may be disposed in any arrangement along or about any number of segments. For example, in some embodiments, the IMUs 210 may be disposed in a linear or regular pattern associated with a particular axis of the segment. As another example, the IMUs 210 may be disposed in a spaced apart manner (e.g., circumferentially or randomly about the segment) to cover an entire surface or portion of a surface of the segment. Additionally or alternatively, the IMUs 210 may be placed in any orientation or distribution about a segment of the user.

Modifications, additions, or omissions may be made to the embodiments illustrated in FIGS. 2A-2D. For example, any number of other components that may not be explicitly illustrated or described may be included. As another example, any number and/or type of sensors may be included and may be arranged in any manner.

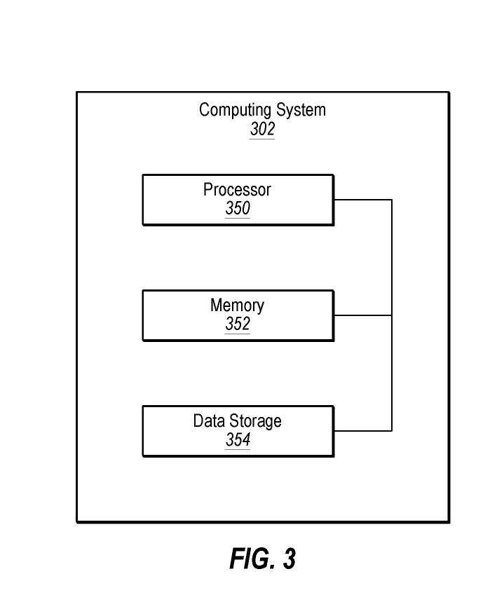

FIG. 3 illustrates a block diagram of an example computing system 302, in accordance with one or more embodiments of the present disclosure. The computing device 120 and/or the computing device 160 may be implemented in a similar manner to the computing system 302. The computing system 302 may include a processor 350, a memory 352, and a data storage 354. The processor 350, the memory 352, and the data storage 354 may be communicatively coupled.

In general, the processor 350 may include any suitable special-purpose or general-purpose computer, computing entity, or processing device including various computer hardware or software modules and may be configured to execute instructions stored on any applicable computer-readable storage media. For example, the processor 350 may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data. Although illustrated as a single processor in FIG. 3, the processor 350 may include any number of processors configured to perform, individually or collectively, any number of operations described in the present disclosure. Additionally, one or more of the processors may be present on one or more different electronic devices, such as different servers.

In some embodiments, the processor 350 may interpret and/or execute program instructions and/or process data stored in the memory 352, the data storage 354, or the memory 352 and the data storage 354. In some embodiments, the processor 350 may fetch program instructions from the data storage 354 and load the program instructions in the memory 352. After the program instructions are loaded into memory 352, the processor 350 may execute the program instructions.

The memory 352 and the data storage 354 may include computer-readable storage media for carrying or having computer-executable instructions or data structures stored thereon. Such computer-readable storage media may include any available media that may be accessed by a general-purpose or special-purpose computer, such as the processor 350.

By way of example, and not limitation, such computer-readable storage media may include tangible or non-transitory computer-readable storage media including RAM, ROM, EEPROM, CD-ROM or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable storage media. Computer-executable instructions may include, for example, instructions and data configured to cause the processor 350 to perform a certain operation or group of operations.

Modifications, additions, or omissions may be made to the computing system 302 without departing from the scope of the present disclosure. For example, in some embodiments, the computing system 302 may include any number of other components that may not be explicitly illustrated or described.

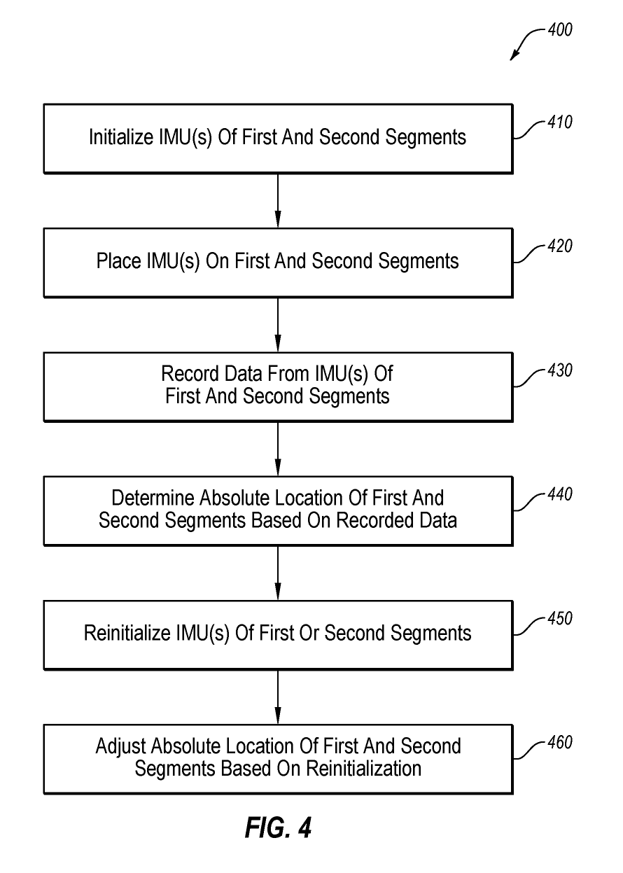

FIG. 4 illustrates a flowchart of an example method 400 for determining biomechanical information of a subject, in accordance with one or more embodiments of the present disclosure. The method 400 may be implemented by any device or system, such as the system 100, the computing device 120, and/or the computing device 160 of FIG. 1, and/or the computing system 302 of FIG. 3.

At block 410, one or more IMUs of a first segment and one or more IMUs of a second segment of a subject may be initialized. For example, IMUs of the first segment may be placed in a calibration tray located at a known location with the IMUs in a particular orientation. The initialization may additionally include pairing or otherwise placing the IMUs in communication with a computing device to capture data generated by the IMUs.

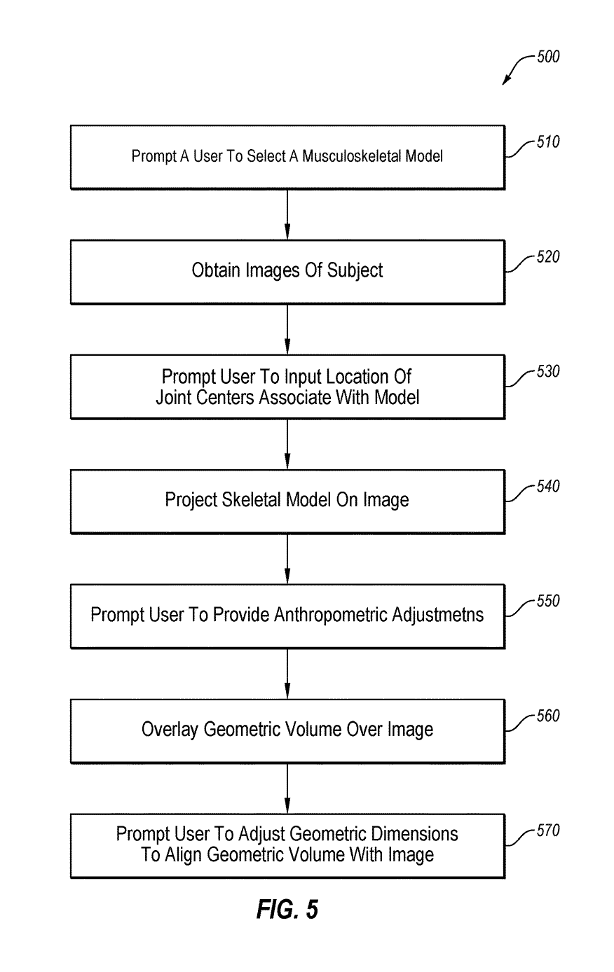

At block 420, the IMUs may be placed on the first segment and the second segment of the subject. For example, the IMUs may be strapped to the subject, or a sleeve or other wearable material with the IMUs coupled thereto may be worn by the subject. In some embodiments, the operation of the block 420 may be performed before the operation of the block 410. For example, the IMUs may be placed upon the first segment and the second segment of the subject, and after the IMUs have been placed upon the subject, images may be captured of the subject and the IMUs by cameras at a known location. Additionally or alternatively, 3D scans may be taken of the subject. In these and other embodiments, initialization may include any number of other steps and/or operations, for example, those illustrated in FIG. 5. In some embodiments, rather than positioning IMUs upon a first and a second segment, IMUs may be placed on only a single segment (e.g., a trunk of a user). In these and other embodiments, information from the IMUs of the single segment may be used on its own or may be coupled with data from one or more sensors measuring force (e.g., a pressure sensor) or physiological data.

At block 430, data may be recorded from the IMUs of the first and second segments. For example, as the subject moves through a series of motions such as walking, standing in a given posture, etc., the IMUs may measure and generate data such as position, velocity, acceleration, etc. and the generated data may be recorded by a computing device. For example, the IMUs 110 of FIG. 1 may generate data that is recorded by the computing device 120 of FIG. 1.

At block 440, the absolute location of the first and segments may be determined based on the recorded data. For example, the computing device 120 of FIG. 1 may determine the absolute location and/or the computing device 120 may communicate the recorded data to the computing device 160 of FIG. 1 and the computing device 160 may determine the absolute location. In some embodiments, determining the absolute location may include extrapolating acceleration information of each of the IMUs to determine velocity and/or position (e.g., by a first and/or second derivative of the acceleration information). Additionally, such a determination may include averaging over multiple IMUs, correcting based on one or more GPS sensors, etc.

At block 450, the IMUs of the first and second segments may be reinitialized. For example, after the subject has performed the series of motions, the IMUs may be placed back in a calibration tray, or additional images may be captured of the subject and the IMUs by an image capturing device at a known location.

At block 460, the absolute location of the first and second segments may be adjusted based on the initialization. For example, if the location of the IMUs at the re-initialization registers different than what the absolute location is determined to be at the block 440, the absolute location determinations may be adjusted and/or corrected based on the re-initialization to the known initialization known location. In some embodiments, other corrections may be performed after the adjustment at the block 460. For example, averaging over multiple IMUs, etc., may be performed after correcting based on the re-initialization.

Modifications, additions, or omissions may be made to the method 400 without departing from the scope of the present disclosure. For example, the operations of the method 400 may be implemented in differing order, such as the block 420 being performed before the block 410. Additionally or alternatively, two or more operations may be performed at the same time. Furthermore, the outlined operations and actions are provided as examples, and some of the operations and actions may be optional, combined into fewer operations and actions, or expanded into additional operations and actions without detracting from the essence of the disclosed embodiments. For example, the blocks 450 and 460 may be omitted. Additionally, other operations may be added, such as determining kinematic information about a joint between the first and second segments, determining other biomechanical information, or monitoring and/or utilizing pressure data in such determinations. As another example, while described as using IMUs, any number of types or other sensors may be used, e.g., sensors for measuring physiological data.

FIG. 5 illustrates a flowchart of an example method for initializing one or more sensors, in accordance with one or more embodiments of the present disclosure. The method 500 may be implemented by any device or system, such as the system 100, the computing device 120, and/or the computing device 160 of FIG. 1, and/or the computing system 302 of FIG. 3.

At block 510, a user may be prompted to select a musculoskeletal model. For example, a user of a computing device (e.g. the computing device 160 of FIG. 1) may be prompted to select or enter a musculoskeletal model (e.g. lower extremity only, lower extremity with torso, full body, Trendelenburg, etc.).

At block 520, images may be obtained of the subject. For example, the image capturing device 150 of FIG. 1 may be used to capture images of the subject. In some embodiments, the image capturing device may be at a fixed known location from which images are captured. In some embodiments, the image capturing device may be movable from a known calibration location to capture images of the subject, whether a video or multiple still images. One or more sensors associated with the subject may also be captured in the images. In some embodiments, each sensor (e.g. an IMU or GPS sensor) may be in two or more images. In some embodiments, 3D scans may be captured in addition to or in place of images.

At block 530, a user may be prompted to input location of joint centers associated with the model selected at the block 510. For example, one or more of the images captured at the block 520 may be displayed to the user and the user may identify the joint centers in the images. For example, the user may use a touch screen, mouse, etc. to identify the joint centers. In some embodiments, a suggested or estimated joint center may be provided to the user and the user may be given the option to confirm the location of the joint center or to modify the location of the joint center. Additionally, the location of one or more of the sensors may be input by the user in a similar manner (e.g., manual selection, confirming a system-provided location, etc.).

At block 540, a skeletal model may be projected on one or more images. For example, for the musculoskeletal model of the block 510, the skeletal components of the musculoskeletal model may be overlaid the image of the subject in an anatomically correct position. For example, the tibia, ulna, and fibula will be projected over the legs of the subject in the image. In some embodiments, the user may be provided with an opportunity to adjust the location and/or orientation of the skeletal model within the image.

At block 550, the user may be prompted to provide anthropometric adjustments. For example, the user may be prompted to input height, weight, age, gender, etc. of the subject. In these and other embodiments, the skeletal model may be adjusted and or modified automatically based on the anthropometric information.

At block 560, one or more geometric volumes may be overlaid the image of the subject. For example, an ellipsoid, frustum, sphere, etc. representing portions of the user may be overlaid on the image. For example, an ellipsoid corresponding to the lower leg may be placed over the image of the lower leg of the subject.

At block 570, the user may be prompted to adjust the geometric dimensions to align the geometric volume with the image. For example, the user may be able to adjust the major axis, minor axis, and/or location of the geometric volume (which may also adjust the skeletal model) such that the edges of the geometric volume correspond with the edges of a segment of the subject. For example, if the segment of interest was a lower leg of the subject and an ellipsoid was overlaid over the lower leg segment, the ellipsoid may be adjusted such that the edges of the ellipsoid aligned with the edges of the lower leg in the image of the subject by adjusting the magnitude of the minor axis and the location of the minor axis along the length of the ellipsoid.

Modifications, additions, or omissions may be made to the method 500 without departing from the scope of the present disclosure. For example, the operations of the method 500 may be implemented in differing order. Additionally or alternatively, two or more operations may be performed at the same time. Furthermore, the outlined operations and actions are provided as examples, and some of the operations and actions may be optional, combined into fewer operations and actions, or expanded into additional operations and actions without detracting from the essence of the disclosed embodiments. For example, the blocks 530, 540, 550, 560, and/or 570 may be omitted. Additionally, other operations may be added, such as obtaining a 3D scan of the subject, identifying an absolute location of an image capturing device, initializing sensors (e.g. IMUs), etc.

As used in the present disclosure, the terms “module” or “component” may refer to specific hardware implementations configured to perform the actions of the module or component and/or software objects or software routines that may be stored on and/or executed by general purpose hardware (e.g., computer-readable media, processing devices, etc.) of the computing system. In some embodiments, the different components, modules, engines, and services described in the present disclosure may be implemented as objects or processes that execute on the computing system (e.g., as separate threads). While some of the system and methods described in the present disclosure are generally described as being implemented in software (stored on and/or executed by general purpose hardware), specific hardware implementations or a combination of software and specific hardware implementations are also possible and contemplated. In this description, a “computing entity” may include any computing system as previously defined in the present disclosure, or any module or combination of modulates running on a computing system.

Terms used in the present disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

All examples and conditional language recited in the present disclosure are intended for pedagogical objects to aid the reader in understanding the disclosure and the concepts contributed by the inventor to furthering the art, and are to be construed as being without limitation to such specifically recited examples and conditions. Although embodiments of the present disclosure have been described in detail, various changes, substitutions, and alterations could be made hereto without departing from the spirit and scope of the present disclosure.

What is claimed is:

1. A method comprising:
recording first initial orientation information of a first inertial measurement unit (IMU) placed in a first initialization position at a first initialization location;
recording second initial orientation information of a second IMU placed in a second initialization position at a second initialization location;
placing the first IMU on a first segment of a subject;
placing the second IMU on a second segment of the subject, wherein the first segment and the second segment move relative to each other about a joint of the subject;
recording first acceleration information output by the first IMU in a continuous manner after recordation of the first initial orientation information of the first IMU;
recording second acceleration information output by the second IMU in a continuous manner after recordation of the second initial orientation information of the second IMU;
determining a first absolute location of the first segment with respect to the first initialization location based on the first acceleration information and the first initial orientation information;
determining a second absolute location of the second segment with respect to the second initialization location based on the second acceleration information and the second initial orientation information; and
determining kinematics of the first segment and the second segment with respect to the joint based on the first absolute location and the second absolute location.

2. The method of claim 1, further comprising:
recording first final orientation information of the first IMU at the first initialization location;
determining a difference between the first final orientation and the first initial orientation information; and
adjusting the first absolute location based on the difference.

3. The method of claim 1, further comprising:
placing a third IMU on the first segment;
recording third acceleration information output by the third IMU; and
wherein determining the first absolute location is further based on the third acceleration information.

4. The method of claim 3, further comprising:
comparing a first determination of the first absolute location based at least on the first acceleration information with a second determination of the first absolute location based at least on the third acceleration information; and
correcting the first absolute location by an offset amount related to the comparison.

5. The method of claim 1, further comprising:
placing a force sensor at a contact point on the subject, the force sensor configured to obtain pressure information with respect to pressure applied to a surface by the contact point; and
wherein the kinematics of the first segment and the second segment with respect to the joint are further based on the pressure information.

6. A system comprising:
a first inertial measurement unit (IMU) attached to a first segment of a subject;
a second IMU attached to a second segment of the subject, wherein the first segment and the second segment move relative to each other about a joint of the subject;
a first force sensor configured to attach to a first contact point of the subject, wherein the first force sensor is configured to attach to the first contact point such that the first force sensor is configured to obtain first pressure information with respect to pressure applied to a surface by the first contact point;
a second force sensor configured to attach to a second contact point of the subject, wherein the second force sensor is configured to attach to the second contact point such that the second force sensor is configured to obtain second pressure information with respect to pressure applied to the surface by the second contact point; and
a computing system communicatively coupled to the first IMU, the second IMU, the first force sensor, and the second force sensor, wherein the computing system is configured to:
obtain first acceleration information measured by the first IMU;
obtain second acceleration information measured by the second IMU;
obtain first pressure information measured by the first force sensor;
obtain second pressure information measured by the second force sensor; and
determine kinetics of the subject with respect to the joint based on the first acceleration information, the second acceleration information, the first pressure information, and the second pressure information.

7. The system of claim 6, wherein the computing system is further configured to determine the kinetics with respect to one or more of the following:
a time when both the first contact point and the second contact point are applying pressure to the surface;
a time when the first contact point is applying pressure to the surface and the second contact point is not applying pressure to the surface; and
a time when the second contact point is applying pressure to the surface and the first contact point is not applying pressure to the surface.

8. The system of claim 6, further comprising a first plurality of IMUs attached to the first segment and a second plurality of IMUs attached to the second segment.

9. A method comprising:
initializing a first plurality of inertial measurement units (IMUs) and a second plurality of IMUs;
attaching the first plurality of IMUs to a first segment of a subject and the second plurality of IMUs to a second segment of the subject;
obtaining data from the first plurality of IMUs and the second plurality of IMUs as the subject performs a motion; and
determining an absolute position of the first segment and the second segment based on the data.

10. The method of claim 9, wherein the first plurality of IMUs are attached to the subject before initializing the first plurality of IMUs.

11. The method of claim 9, wherein initializing the first plurality of IMUs comprises:
obtaining a plurality of images, each of the first plurality of IMUs being in one or more of the plurality of images;
displaying at least one of the plurality of images;
identifying one or more joints of the subject in the at least one of the plurality of images;
projecting a skeletal model over the subject in the at least one of the plurality of images; and
overlaying a geometric shape over the at least one of the plurality of images, the geometric shape corresponding to the first segment.

12. The method of claim 11, further comprising:
providing a prompt to identify one or more joints of the subject in the at least one of the plurality of images; and
receiving an identification of one or more joints of the subject.

13. The method of claim 11, further comprising:
providing a prompt to input anthropometric information; and
receiving anthropometric information of the subject;
wherein at least one of the skeletal model and the geometric shape is based on the anthropometric information of the subject.

14. The method of claim 11, further comprising:
providing a prompt to adjust the geometric shape to align the geometric shape with an outline of the subject;
receiving an input to adjust the geometric shape; and
adjusting the geometric shape based on the input.

15. The method of claim 11, further comprising:
obtaining global positioning system (GPS) location of an image capturing device; and
capturing at least one of the plurality of images using the image capturing device.

16. The method of claim 15, further comprising:
placing the image capturing device in a fixed location of a known position; and
wherein the GPS location of the image capturing device is the fixed location.

17. The method of claim 15, wherein capturing at least one of the plurality of images comprises:
capturing a plurality of images using a plurality of image capturing devices such that each IMU of the first plurality of IMUs is in at least two of the plurality of images.

18. The method of claim 15, wherein capturing at least one of the plurality of images comprises capturing a video of the subject, the video capturing each of the first plurality of IMUs.

19. The method of claim 15, further comprising
determining an image-based absolute position of the first segment based on the GPS location of the image capturing device; and
modifying the absolute position based on the image-based absolute position.

20. The method of claim 9, wherein initializing the IMUs includes performing a three-dimensional scan of the subject.
